(19) United States
(12) Patent Application Publication
(10) Pub. No.: US 2024/0130798 A1
(43) Pub. Date: Apr. 25, 2024

(54) SYSTEMS AND METHODS FOR GUIDED AIRWAY CANNULATION

(71) Applicants: Massachusetts Institute of Technology, Cambridge, MA (US); US Army Institute of Surgical Research, Houston, TX (US)

(72) Inventors: Laura Brattain, Lexington, MA (US); Brian A. Telfer, Lincoln, MA (US); Matthew Johnson, Medford, MA (US); Lars Gjesteby, Maynard, MA (US); Joshua Werblin, Boston, MA (US); Gregory R. Dion, San Antonio, TX (US); Ronit E. Malka, San Antonio, TX (US); Shawn Moynihan, Cambridge, MA (US)

(21) Appl. No.: 18/217,555

(22) Filed: Jun. 30, 2023

Related U.S. Application Data
(60) Provisional application No. 63/357,911, filed on Jul. 1, 2022.

(51) Int. Cl. A61B 34/20; A61B 8/08; A61M 25/01; G06T 7/73

(52) U.S. Cl. CPC A61B 34/20; A61B 8/0841; A61M 25/01; G06T 7/73; A61B 2034/2063; G06T 2207/10132; G06T 2207/20081; G06T 2207/20084

(57) ABSTRACT

Systems and methods are provided for semi-automated, portable, ultrasound guided cannulation. The systems and methods provide for image analysis to provide for identification of anatomical landmarks from image data. The image analysis provides for guidance for insertion of a cannulation system into an airway of a subject which may be accomplished by a non-expert based upon the guidance provided. The system further enables a single person to perform the cannulation rather than the typical 2 or more people. The guidance may include an indicator or a mechanical guide to guide a user for inserting the cannulation system into a subject to penetrate the airway of interest.

### CROSS-REFERENCE TO RELATED APPLICATIONS

This application is based on, claims priority to, and incorporates herein by reference U.S. Provisional Application Ser. No. 63/357,911, filed Jul. 1, 2022.

### STATEMENT REGARDING FEDERALLY SPONSORED RESEARCH

This invention was made with government support under FA8702-15-D-0001 awarded by the U.S. Army and Defense Health Agency. The government has certain rights in the invention.

### BACKGROUND

Insertion of catheters into blood vessels, veins, or arteries can be a difficult task for non-experts or in trauma applications because the vein or artery may be located deep within the body, may be difficult to access in a particular patient, or may be obscured by trauma in the surrounding region to the vessel. Multiple attempts at penetration may result in extreme discomfort to the patient, loss of valuable time during emergency situations, or in further trauma. Furthermore, central veins and arteries are often in close proximity to each other. While attempting to access the internal jugular vein, for example, the carotid artery may instead be punctured, resulting in severe complications or even mortality due to consequent blood loss due to the high pressure of the blood flowing in the artery. Associated nerve pathways may also be found in close proximity to a vessel, such as the femoral nerve located nearby the femoral artery, puncture of which may cause significant pain or loss of function for a patient.

To prevent complications during cannulation, ultrasonic instruments can be used to determine the location and direction of the vessel to be penetrated. One method for such ultrasound guided cannulation involves a human expert who manually interprets ultrasound imagery and inserts a needle. Such a manual procedure works well only for experts who perform the procedure regularly so that they may accurately cannulate a vessel.

Systems have been developed in an attempt to remove or mitigate the burden on the expert, such as robotic systems that use a robotic arm to insert a needle. These table-top systems and robotic arms are too large for portable use, such that they may not be implemented by medics at a point of injury. In addition, these systems are limited to peripheral venous access, and may not be used to cannulate more challenging vessels or veins.

Still other systems have been used to display an image overlay on the skin to indicate where a vessel may be located, or otherwise highlight where the peripheral vein is located just below the surface. However, in the same manner as above, these systems are limited to peripheral veins, and provide no depth information that may be used by a non-expert to guide cannulation, not to mention failures or challenges associated with improper registration.

Cricothyrotomy and tracheotomy are two surgical procedures that allow a patient's airway to be accessed through the neck when a patient cannot breathe and endotracheal intubation (through the mouth or nose) is not possible or applicable. A cricothyrotomy is an emergency procedure in which a breathing tube is inserted into the trachea through the cricothyroid membrane (between the thyroid cartilage and cricoid cartilage that are key anatomical landmarks). A tracheotomy is typically performed in a hospital operating room (OR) or intensive care unit (ICU) by inserting a breathing tube into the trachea below the cricoid cartilage. Cricothyrotomy is a temporizing measure. Patients who undergo cricothyrotomy typically need to be converted to tracheotomy to avoid long-term complications.

Overall, about 100,000 tracheotomies are normally performed in the U.S. each year, although the number has increased during the COVID-19 pandemic. Emergency cricothyrotomy is not performed often but is a critical, lifesaving procedure. It currently suffers from high failure rate due to the fact that it requires repeated training to gain and maintain experience.

Several commercial products assist a user in performing a cricothyrotomy, such as the QuickTrach2. These products are intended to simplify inserting the breathing tube. However, these devices do not address the primary causes of failed cricothyrotomies, which are that the breathing tube is incorrectly inserted outside of the trachea, either above it or to the side.

Many of these procedures could benefit from enhanced guidance. Therefore, there is a need for techniques for improved cannulation of airway passages that is less cumbersome, more accurate, and able to be deployed by a non-expert.

### SUMMARY OF THE DISCLOSURE

The present disclosure addresses the aforementioned drawbacks by providing new systems and methods for guided airway cannulation. The systems and methods provide for image analysis to provide for segmentation of airway passages of interest from image data. The image analysis provides guidance for insertion of a cannulation system into a subject and may be accomplished by a non-expert based upon the guidance provided. The guidance may include an indicator or a mechanical guide to guide a user when inserting the cannulation system into a subject to penetrate the airway of interest.

In one configuration, a system is provided for guiding an interventional device in an interventional procedure of a subject. The system includes an ultrasound probe and a guide system coupled to the ultrasound probe and configured to guide the interventional device into a field of view (FOV) of the ultrasound probe. The system also includes a non-transitory memory having instructions stored thereon. The system also includes a processor configured to access the non-transitory memory and execute the instructions. The processor is configured to access image data acquired from the subject using the ultrasound probe; the image data include at least one image of an anatomical landmark structure of the subject. The processor is also configured to determine, from the image data and the anatomical landmark structure, a location of a target airway within the subject. The processor is also configured to determine an insertion point location for the interventional device based upon the location of the target airway and guide placement of the ultrasound probe to position the guide system at the insertion point location and track the interventional device from the insertion point location to the target structure.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a schematic diagram of a non-limiting example ultrasound system that can implement the systems and methods described in the present disclosure.

FIG. 2 is a schematic diagram of a non-limiting example configuration for guiding an insertion device into an airway of interest using an ultrasound probe.

FIG. 3 is a flowchart of non-limiting example steps for a method of operating a system for guiding airway cannulation.

FIG. 4A is another flowchart of non-limiting example steps for a method of operating a system for guiding airway cannulation.

FIG. 4B is a flowchart of non-limiting example steps for a method of guiding penetration of an airway or vessel of interest.

FIG. 4C is a flowchart of non-limiting example steps for a method of AI-guided penetration of an airway of interest.

FIG. 5A is a block diagram of an example system that can implement an airway of interest image processing system for generating images of an airway of interest and anatomical landmarks or otherwise providing insertion guidance for an airway of interest using a hybrid machine learning and mechanistic model.

FIG. 5B is a block diagram of example hardware components of the system of FIG. 5A.

FIG. 6A is a block diagram of a non-limiting example machine learning or artificial intelligence (AI) architecture.

FIG. 6B shows an ROC curve of a frame-level classification model according to aspects of the present disclosure.

FIG. 6C is an example of frame-level classification utilized by machine learning or AI, according to aspects of the present disclosure. Frames are shown predicting anatomical landmarks based on the probe location on a subject's neck.

FIG. 6D is an example of bounding box detection utilized by machine learning or AI, according to aspects of the present disclosure. Frames are shown with bounding boxes identifying anatomical landmarks in acquired images.

FIG. 7A is a perspective view of a non-limiting example interventional device guide coupled to an ultrasound probe.

FIG. 7B is a side view of the interventional device guide of FIG. 7A.

FIG. 7C is a side view of the base and ultrasound probe fixture for the interventional device guide of FIG. 7B.

FIG. 7D is a cross-section of a non-limiting example cartridge compatible with the injection assemble of FIG. 7B.

FIG. 7E is a side view of a non-limiting example interventional device guide penetration assembly.

FIG. 7F is a perspective view of the device of FIG. 7E.

FIG. 7G is a lower view of the device of FIG. 7E.

FIG. 7H is a perspective view of a non-limiting example interventional device guide penetration assembly with an optical stabilizer.

FIG. 8A is a perspective view of a non-limiting example interventional device guide integrated with an ultrasound probe.

FIG. 8B is an exploded view of the integrated interventional device guide and ultrasound probe of FIG. 8A.

FIG. 9 is perspective view of a non-limiting example cricothyrotomy cartridge for use in accordance with the present disclosure.

FIG. 10A is a side view of inserting a non-limiting example dilating component into the interventional device guide.

FIG. 10B is a side view of aligning the non-limiting example dilating component with the interventional device guide and advancing a needle to guide the non-limiting example dilating component into the subject.

FIG. 10C is a side view of advancing the non-limiting example dilating component over the needle and into the subject.

FIG. 10D is a side view of retracting the needle and leaving the non-limiting example dilating component in the subject.

FIG. 10E is a side view of removing the interventional device guide and leaving the non-limiting example dilating component in the subject.

FIG. 11A is a side view of a non-limiting example needle/cannula advancement into a subject.

FIG. 11B is a side view of a non-limiting example dilation advancement into a subject.

FIG. 11C is a side view of a non-limiting example of a dilator retraction.

FIG. 11D is a side view of a non-limiting example trach engagement.

FIG. 11E is a side view of a non-limiting example trach advancement.

FIG. 12A is a side view of a non-limiting example of an interventional device within a cartridge.

FIG. 12B is a side view of a non-limiting example of an interventional device.

FIG. 12C is a front view of a non-limiting example of the international device of FIG. 12B.

FIG. 13A is a side view of a non-limiting example needle/cannula advancement into a subject.

FIG. 13B is a side view of a non-limiting example of blade and dilation advancement into a subject.

FIG. 13C is a side view of a non-limiting example of a needle and blade retraction.

FIG. 13D is a side view of a non-limiting example dilator position within the trachea and catheter advancement.

FIG. 13E is a side view of a non-limiting example endotracheal tube advancement.

### DETAILED DESCRIPTION

Systems and methods are provided for guided airway cannulation. The systems and methods provide for image analysis and machine learning to provide for segmentation of airway passages of interest from image data and for guidance of a cannulation procedure. The image analysis provides guidance for insertion of a cannulation system into a subject and may be accomplished by a non-expert based upon the guidance provided. The guidance may include an indicator or a mechanical guide to guide a user when inserting the cannulation system into a subject to penetrate the airway of interest

A machine learning, or artificial intelligence (AI) guided airway cannulation system or “AI-GUIDE-Airway” may be used is to assist medical providers in performing surgical airway procedures more efficiently, more accurately, more safely, and with less exposure to potentially contagious aerosols. Surgical airway procedures may include cricothyrotomy, tracheotomy and the like. In the case of efficiency, a single provider may be enabled to perform a tracheotomy using the systems and methods of the present disclosure instead of the three or more providers that are needed for conventional procedures. In the case of accuracy and safety, one-third of emergency cricothyrotomies fail due to improper placement and inability to locate and access the airway. An AI-GUIDE-Airway procedure may significantly reduce that error.

For tracheotomy, percutaneous procedures, which start with a needle insertion through the skin, have become more common compared to the traditional more complex open procedure, but are not appropriate for all patients, such as those with morbid obesity, challenging neck anatomy, who are on blood thinners or who need an emergency airway. These cases represent perhaps 20% of the patients that need a tracheotomy. The increased accuracy and safety provided by a guided airway cannulation procedure may improve patient outcomes and to allow more of these patients to more promptly receive a tracheotomy in the ICU rather than the OR, which also reduces cost. With automated neck ultrasound interpretation and guidance, a guided cannulation in accordance with the present disclosure may bridge the training and experience gap.

Additionally, surgical airway access generates aerosols. In the setting of emerging viral illnesses such as COVID-19, potential aerosol exposure to hospital personnel can result in altered treatment patterns, e.g., reduced number of procedures, to protect providers. A guided airway cannulation in accordance with the present disclosure may operate under an integrated protective barrier, which significantly reduces an operator's exposure to aerosols.

In some configurations, successful airway access may be provided by a machine learning or AI method that identifies key neck landmarks, such as thyroid cartilage, cricothyroid membrane (CTM), cricoid cartilage, thyroid gland, tracheal rings, and the like. Proper tube insertion location may be determined using automated image interpretation from neck ultrasound images acquired by an ultrasound system, such as a commercial ultrasound system. In some configurations, a handheld robotic module, integrated with a commercial ultrasound probe, may be used to perform a series of steps to insert a breathing tube. A machine learning or AI system, software, and/or embedded hardware sensor, may be used to confirm proper tube placement. Automated neck ultrasound sensing and interpretation may be used in procedures such as cricothyrotomy and tracheotomy, which are currently performed using manual palpation and manual incisions.

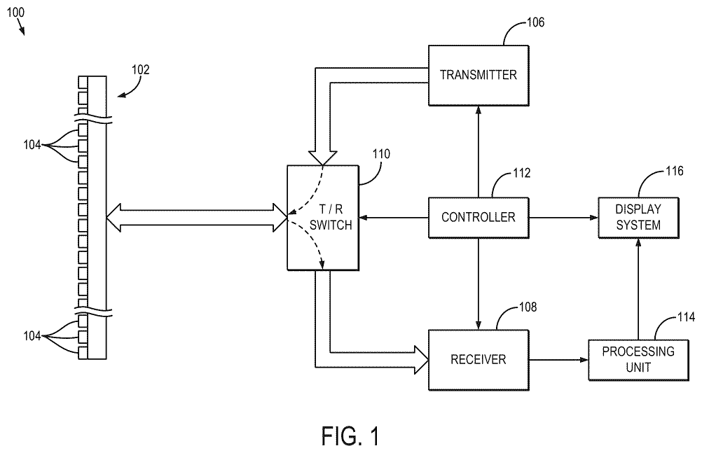

FIG. 1 illustrates an example of an ultrasound system 100 that can implement the methods described in the present disclosure. The ultrasound system 100 includes a transducer array 102 that includes a plurality of separately driven transducer elements 104. The transducer array 102 can include any suitable ultrasound transducer array, including linear arrays, curved arrays, phased arrays, and so on. Similarly, the transducer array 102 can include a 1D transducer, a 1.5D transducer, a 1.75D transducer, a 2D transducer, a 3D transducer, and so on.

When energized by a transmitter 106, a given transducer element 104 produces a burst of ultrasonic energy. The ultrasonic energy reflected back to the transducer array 102 (e.g., an echo) from the object or subject under study is converted to an electrical signal (e.g., an echo signal) by each transducer element 104 and can be applied separately to a receiver 108 through a set of switches 110. The transmitter 106, receiver 108, and switches 110 are operated under the control of a controller 112, which may include one or more processors. As one example, the controller 112 can include a computer system.

The transmitter 106 can be programmed to transmit unfocused or focused ultrasound waves. In some configurations, the transmitter 106 can also be programmed to transmit diverged waves, spherical waves, cylindrical waves, plane waves, or combinations thereof. Furthermore, the transmitter 106 can be programmed to transmit spatially or temporally encoded pulses.

The receiver 108 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 106 and the receiver 108 can be programmed to implement a high frame rate. For instance, a frame rate associated with an acquisition pulse repetition frequency (“PRF”) of at least 100 Hz can be implemented. In some configurations, the ultrasound system 100 can sample and store at least one hundred ensembles of echo signals in the temporal direction.

The controller 112 can be programmed to implement an imaging sequence using the techniques described in the present disclosure, or as otherwise known in the art. In some embodiments, the controller 112 receives user inputs defining various factors used in the design of the imaging sequence.

A scan can be performed by setting the switches 110 to their transmit position, thereby directing the transmitter 106 to be turned on momentarily to energize transducer elements 104 during a single transmission event according to the implemented imaging sequence. The switches 110 can then be set to their receive position and the subsequent echo signals produced by the transducer elements 104 in response to one or more detected echoes are measured and applied to the receiver 108. The separate echo signals from the transducer elements 104 can be combined in the receiver 108 to produce a single echo signal.

The echo signals are communicated to a processing unit 114, which may be implemented by a hardware processor and memory, to process echo signals or images generated from echo signals. As an example, the processing unit 114 can guide cannulation of a vessel of interest using the methods described in the present disclosure. Images produced from the echo signals by the processing unit 114 can be displayed on a display system 116.

In some configurations, a non-limiting example method may be deployed on an imaging system, such as a commercially available imaging system, to provide for a portable ultrasound system with airway cannulation guidance. The systems and methods may locate an airway passage, and may provide real-time guidance to the user to position the ultrasound probe and airway cannulation device to the optimal insertion point. The systems may determine a rotational angle for the ultrasound probe with respect to the subject. The probe may include one or more of a fixed needle guide device, an adjustable mechanical needle guide, a displayed-image needle guide, and the like. An adjustable guide may include adjustable angle and/or depth. The system may guide or communicate placement or adjustments for the guide for the interventional device, such as a needle. For example, a processor of the system disclosed may determine an angle for the interventional device from an insertion point location to a target airway. The system may also determine or regulate the needle insertion distance from the insertion point location to the target airway based upon the depth computed for the anatomical landmark structure. The user may then insert a needle or cannula through the mechanical guide attached to the probe or displayed guide projected from the probe in order to ensure proper insertion. During insertion, the system may proceed to track the target airway and the penetration device until the airway is penetrated while providing real-time feedback to a user based on tracking the penetration device. A graphical user interface may be used to allow the medic to specify the desired airway and to provide feedback to the medic throughout the process.

For the purposes of this disclosure and accompanying claims, the term “real-time” or related terms are used to refer to and defined a real-time performance of a system, which is understood as performance that is subject to operational deadlines from a given event to a system's response to that event. For example, a real-time extraction of data and/or displaying of such data based on acquired ultrasound data may be one triggered and/or executed simultaneously with and without interruption of a signal-acquisition procedure.

In some configurations, the system may automate all ultrasound image interpretation and insertion computations, while a medic or a user may implement steps that require dexterity, such as moving the probe and inserting the cannula. Division of labor in this manner may avoid using a dexterous robot arm and may result in a small system that incorporates any needed medical expertise.

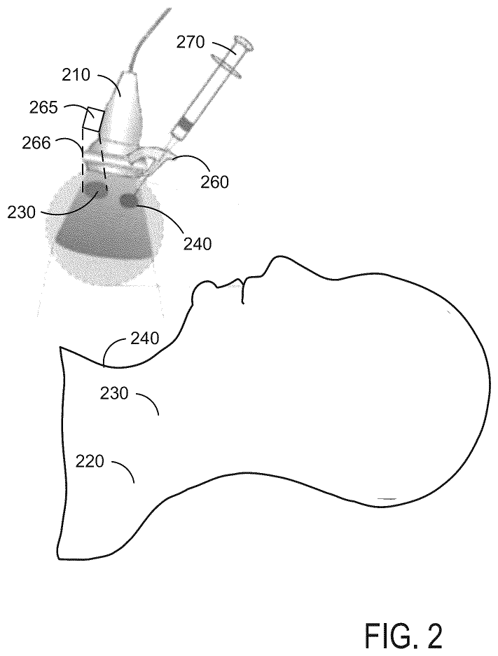

Referring to FIG. 2, a diagram is shown depicting a non-limiting example embodiment for guiding needle insertion into an airway passage 230 of a subject's neck 240. An ultrasound probe 210 is used to acquire an image of a region of interest that includes a portion of the airway passage 230, and any anatomical landmarks 220. The location of the airway passage 230 and/or the anatomical landmark 220 may be annotated on the image. A mechanical guide 260 may be included to guide a needle, dilator, or cannula 270 to penetrate the airway of interest, such as to perform a cricothyrotomy or tracheotomy, and the like. In some configurations, a visual guide 265 may be included where a penetration guide image 266 is projected onto the surface of a subject to guide a needle/dilator/cannula 270 to penetrate the airway of interest. Penetration guide image 266 may reflect the actual size or depth of the airway of interest for penetration when projected onto the subject, or may provide other indicators such as measurements or a point target for penetration, and the like.

Non-limiting example applications may include aiding a medic in performing additional emergency needle insertion procedures, such as needle decompression for tension pneumothorax (collapsed lung) and needle cricothyrotomy (to provide airway access). Portable ultrasound may be used to detect tension pneumothorax and needle insertion point (in an intercostal space, between ribs) or to detect the CTM and needle insertion point.

Anatomical landmarks 220, such as neck landmarks, may be identified along with a proper insertion location. In some configurations, a user may scan an airway identification and cannulation device along a supine patient's neck, starting from just below the chin and moving toward the collarbone. During the course of the scan, images may be processed, such as with a machine learning or AI routine, to automatically recognize the thyroid cartilage, then pass over the small, often less than 1 cm wide CTM, followed by the cricoid cartilage. The thyroid gland that lies to either side of the trachea below the cricoid cartilage may be recognized as a landmark to avoid, as may be significant blood vessels such as the anterior jugular vein. Tracheal rings may be identified as landmarks.

Based on the ultrasound images collected during the scan, a user may be guided via a display with directional arrows back to a proper insertion point for either a cricothyrotomy or tracheotomy. In some configurations, the guidance may be individualized to a particular patient's anatomy. The guidance may be configured to overcome challenges in patient variation, such as the variability in neck anatomy ranging from a long neck and prominent thyroid cartilage, to short muscular necks; the variability in ultrasound images of the trachea, which are air-filled but may contain significant fluid in an injured patient; the difficulty in keeping the ultrasound probe centered on the trachea due to protruding cartilage; the difficulty in detecting the small CTM for cricothyrotomy insertion, and the need to avoid the thyroid gland and critical blood vessels.

In some configurations, a handheld robotic module may be configured to take up less space, which may be exploited to fit to the limited space under the chin. Mechanical neck guides may be configured to fit on varying neck sizes in order to guide the ultrasound scan and to stabilize the neck and trachea while an intubation tube is inserted. A handheld robotic module may be used to perform a sophisticated sequence to insert the breathing tube, starting by inserting a needle and incising the skin, followed by a dilation sheath that is inserted along the needle shaft to create an opening sufficiently large for the breathing tube. The dilator is then retracted, leaving in place a track over which the breathing tube is inserted. In some configurations, the dilator may be configured as the breathing tube. A handheld robotic module may allow for one-person operation, in contrast to the three or more medical care providers that are needed to perform a tracheotomy currently.

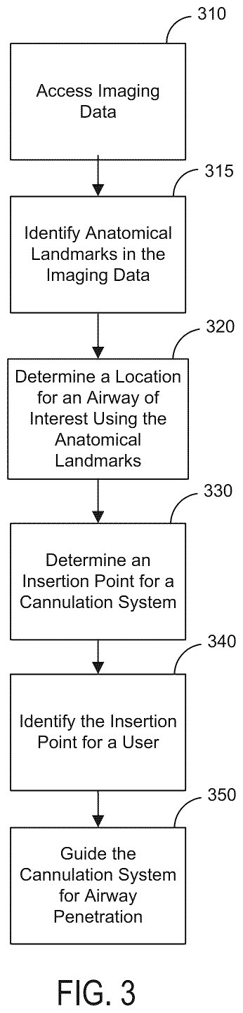

Referring to FIG. 3, provides non-limiting example steps of a method of operating a system for guiding airway cannulation. At step 310, imaging data is accessed. This may be achieved by performing an imaging acquisition in real-time and/or accessing pre-acquired image data. Imaging data may include ultrasound data, and/or may include any other form of medical imaging data, such as magnetic resonance imaging (MRI), computed tomography (CT), PET, SPECT, fluoroscopy, and the like. Using the imaging data, anatomical landmarks may be identified at step 315. Anatomical landmarks, such as neck landmarks, may include a chin, collarbone, thyroid cartilage, CTM, cricoid cartilage, thyroid gland, blood vessels such as the anterior jugular vein, tracheal rings, and the like. In a non-limiting example, a processor is configured to receive a plurality of images of the anatomical landmark structure of the subject acquired in real-time to access the image data. Additionally, the plurality of images may include a plurality of views of the target airway and at a plurality of different timeframes.

Using the imaging data and the identified anatomical landmarks, an airway of interest may be determined at step 320. In a non-limiting example, a processor is configured to assess the plurality of images of the anatomical landmark structure and the plurality of views of the target airway to identify a location on the subject. The location may be determined by segmenting the airway of interest in the imaging data or in using anatomical landmarks for localizing the airway. An insertion point may then be determined at step 330 for an airway cannulation system. Determining the insertion point may be based upon the determined location for the airway of interest and calculating a depth and a pathway for the cannulation system from the surface of a subject to the airway of interest without the cannulation system penetrating other critical structures or organs of interest, such as a nerve.

The insertion point may be determined for a user at step 340. The insertion point may be identified by illuminating a portion of the surface of a subject, or by adjusting a mechanical guide to the appropriate settings for the user, and the like. Depth of the penetration may also be controlled by a setting or a height of the mechanical guide. The airway cannulation system may be guided to the airway of interest for penetration at step 350. Guiding the cannulation system may include acquiring images of the airway of interest and the anatomical landmarks as the cannulation system is inserted into the subject and displaying the tracked images for the user.

A machine learning or AI system may be used to confirm successful insertion of a needle, or cannula, or dilator. Successful insertion may be determined may assessing breathing tube placement using CO₂ sensing and/or ultrasound imaging. If CO₂ is detected, then successful penetration of the airway may be confirmed. For ultrasound imaging, the machine learning or AI system may segment an airway passage wall to determine if the inserted needle, cannula, or dilator has penetrated the airway passage wall, and thereby confirm successful insertion into the airway.

In some configurations, the system can be integrated with a disposable, negative pressure barrier. Non-limiting example negative pressure barriers include a polymer barrier, such as a plastic material, a filter barrier, a HEPA barrier, and the like to isolate the sterile device, such as the sterile tracheotomy device, from the medical personnel operating the system. A negative pressure barrier may prevent spread of aerosolized blood during a surgical procedure by pulling aerosolized particles out of the air or region around the subject. This may free up one hand of the user for manipulation of the endotracheal tube that is in place before tracheotomy placement, adjusting the ventilator circuit, and other tasks, which usually require assistance from additional personnel.

Early tracheotomy, performed less than two weeks after beginning mechanical ventilation, has been recognized as a mechanism to decrease ICU length of stay, improve 90-day mortality, shorten ventilator requirement time, and decrease overall hospital costs in patients requiring prolonged mechanical ventilation. An automated airway penetration system has the potential to be widely used to perform and expand the indications for percutaneous tracheotomies in hospital, as a result of improved efficiency and safety, cost saving, improved patient outcomes, and broader indications for use.

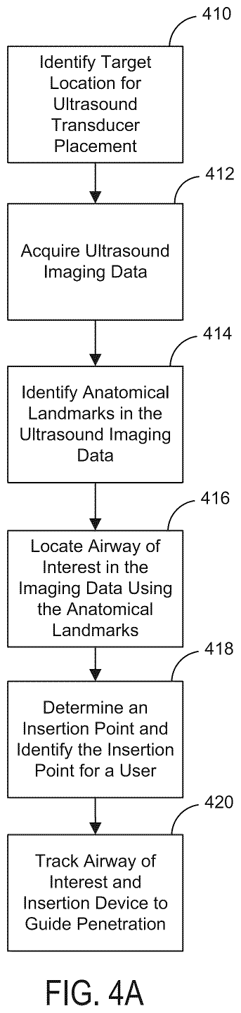

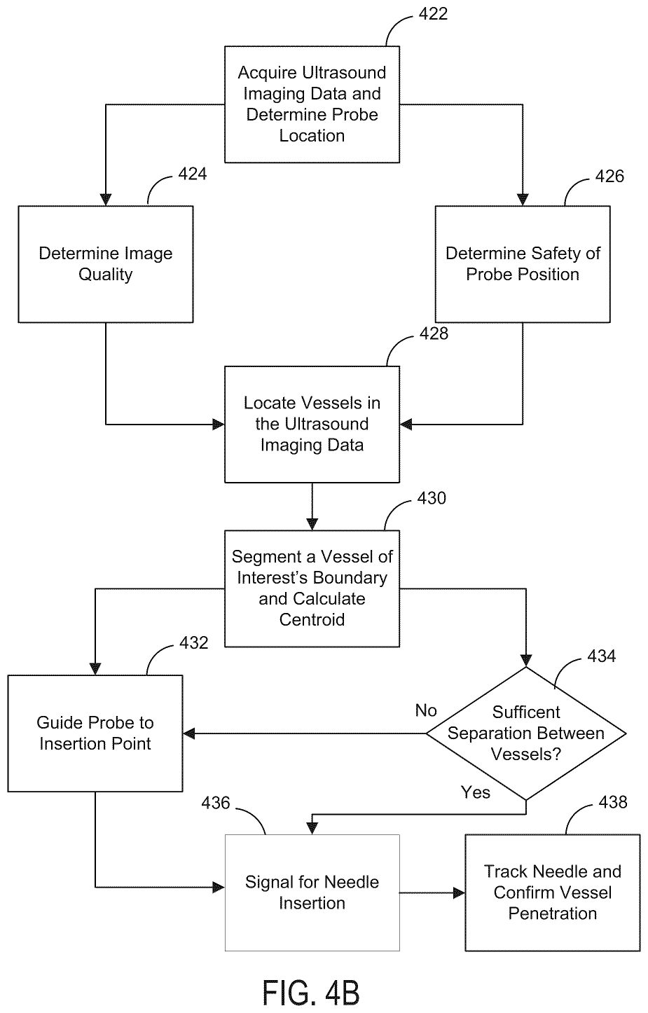

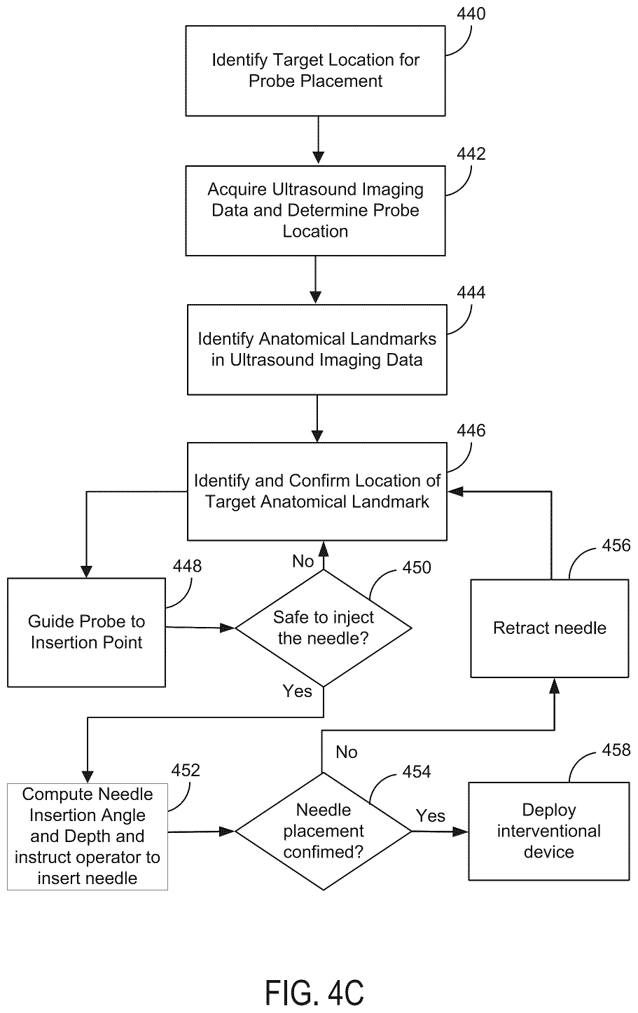

Referring to FIG. 4A, non-limiting example steps are shown in another flowchart setting forth a method of guiding airway cannulation. A target location or region for ultrasound transducer placement may be identified by the system as having been reached at step 410. Ultrasound imaging data may be acquired at step 412 from the target location or region. Anatomical landmarks in the ultrasound imaging data may be identified at step 414. A location for an airway of interest in the imaging data may be determined at step 416 using the identified anatomical landmarks. Identifying an airway of interest using anatomical landmarks may include moving a combined imaging and cannulation device along a portion of a subject's anatomy, such as along a supine patient's neck, starting from just below the chin and moving toward the collarbone. Images may be acquired during the course of the scan and may be processed, such as with a machine learning or AI system, to automatically recognize anatomical landmarks, such as the thyroid cartilage, CTM, cricoid cartilage, and the like. The thyroid gland that lies to either side of the trachea below the cricoid cartilage may be recognized as a landmark to avoid, as may be significant blood vessels such as the anterior jugular vein. Tracheal rings may be identified as landmarks. The anatomical landmarks and any landmarks to avoid may provide guidance for a location of the airway of interest using an understanding of relative locations of the anatomy. The anatomical landmarks and any landmarks to avoid may also provide guidance for how a cannulation device may penetrate from the skin surface of the subject to the airway of interest without penetrating any landmarks to avoid.

An insertion point may then be determined at step 418 for a needle, cannula, or dilator. Determining the insertion point may be based upon the determined and location for the airway of interest and the anatomical landmarks or landmarks to avoid. In some configurations, the method includes calculating a depth and a pathway from the skin surface of a subject to the airway of interest without the needle, cannula, or dilator penetrating device penetrating other organs or structures of interest along the pathway, such as a nerve or landmark to avoid. The insertion point may also be identified for a user at step 418. As above, the insertion point may be identified by illuminating a portion of the surface of a subject, by ensuring a fixed penetration guide is placed over the insertion point, or by automatically adjusting an adjustable mechanical guide to the appropriate settings for the user, and the like. Depth of the penetration may also be controlled by an adjusted setting for the adjustable mechanical guide, or a fixed height of the fixed guide. The needle, cannula or dilator may be tracked and guided to the airway of interest for penetration at step 420. Guiding the device may include acquiring ultrasound images of the airway of interest and the device as the device is inserted into the subject and displaying the tracked images for the user.

Any ultrasound probe may be used in accordance with the present disclosure, including 1D, 2D, linear, phased array, and the like. In some configurations, an image is displayed for a user of the airway of interest with any tracking information for the penetrating device overlaid on the image. In some configurations, no image is displayed for a user and instead only the insertion point may be identified by illuminating a portion of the surface of a subject. In some configurations, no image is displayed and the user is only informed of the probe reaching the proper location whereby a mechanical guide is automatically adjusted to the appropriate settings, such as angle and/or depth to target an airway of interest. The user may be informed of the probe reaching the proper location by any appropriate means, such as light indicator, a vibration of the probe, and the like.

In some configurations, identification of placement of the ultrasound transducer at a target location may be performed automatically by the system at step 410. Image data may be used for identifying anatomical landmarks, such as those described above, and may be accessed by the system to provide automatic identification for where the ultrasound transducer has been placed. In some configurations, a user may specify the airway of interest to be targeted. In a non-limiting example combination of the configurations, the location of the ultrasound transducer on the subject may be automatically determined along with the anatomy being imaged, with the user specifying the airway of interest to target in the automatically identified anatomy. A minimum of user input may be used in order to mitigate the time burden on a user.

Locating the airway of interest at step 416 may be based on machine learning of morphological and spatial information in the ultrasound images. In some configurations, a neural network may be deployed for machine learning and may learn features at multiple spatial and temporal scales. Airways of interest may be distinguished based on shape and/or appearance of the airway, shape and/or appearance of surrounding tissues, relative locations of the anatomical landmarks, and the like. Real-time airway identification may be enabled by a temporally trained routine without a need for conventional post-hoc processing.

Temporal information may be used with locating the airway of interest at step 440. Airway appearances and shape may change with movement of the anatomy over time, such as changes with heartbeat, or differences in appearance between hypotensive and normal-tensile situations. Machine learning routines may be trained with data from multiple time periods with differences in anatomy being reflected over the different periods of time. With a temporally trained machine learning routine, airway identification may be performed in a robust manner over time for a subject without misclassification and without a need to find a specific time frame or a specific probe position to identify vessels of interest.

In some configurations, to prevent any potential misclassifications conflicting information checks may be included in the system. A conflicting information check may include taking into consideration the general configuration of the anatomy at the location of the probe.

Identifying an insertion point for a user at step 418 may also include where the system automatically takes into account the orientation of the probe on a body. A conventional ultrasound probe includes markings on the probe to indicate the right vs left side of probe, which allows a user to orient a probe such that the mark is on the right of the patient, for example. The probe orientation may also be determined from an analysis of the acquired ultrasound images, or monitoring of the orientation of the markings, such as by an external camera. In some configurations, the penetration guide attachment may be configured to fit into the markings on the probe to ensure that the device is consistent with the orientation of the probe.

A safety check may also be performed as part of determining an insertion point at step 418. A safety check may include confirming that there are no critical structures, such as a bone, an unintended blood vessel, a non-target organ, a nerve, and the like, intervening on the path to penetrate the airway. The safety check may also include forcing the system to change the location of the penetration to avoid penetrating such critical structures or landmarks to avoid. In some configurations, the safety check may include confirming the needle has penetrated the airway of interest by the tracking and guidance at step 420, such as by detecting if CO₂ is present after penetration. The safety check may also include determining that the user is holding the system in a stable position, by verifying from the ultrasound image or from an inertial measurement unit on the handle of the system.

Referring to FIG. 4B, non-limiting example steps are shown in a flowchart setting forth a method of guiding needle penetration of a vessel of interest. Ultrasound imaging data is acquired and a probe location is determined at step 422. An image quality may be determined at step 424, and the safety of the probe location for penetrating a vessel in the subject may be determined at step 426. Vessels may be located in the imaging data at step 428. A vessel of interest's boundary may be segmented and a centroid calculated for the vessel of interest at step 430. The probe may be guided to an insertion point at step 432. Sufficient separation between vessels may be determined or confirmed at step 434. If there is not sufficient separation, the probe may be guided to a new insertion position at step 432. If there is sufficient separation, then a signal may be provided to a user to proceed with needle insertion at step 436. Such a signal may be provided on a graphical user interface, or a light in the probe, and the like. The needle may be tracked and vessel penetration confirmed at step 438.

In some configurations, the method includes guiding a user in placement of the ultrasound probe on the subject. A target for penetration may be identified, such as by machine learning in accordance with the present disclosure, and localized. A user may then be guided in which direction to move the ultrasound probe for placement over an identified target. Once the ultrasound probe has reached the target location, a signal may indicate for the user to stop moving the probe. Guidance may be provided by the signal, such as the light on the probe, in a non-limiting example. Needle placement and penetration may proceed after the location of the target has been reached.

Referring to FIG. 4C, non-limiting example steps are shown in a flowchart setting forth an AI guided method for inserting an interventional device in an airway of interest. A target location for probe placement is identified by the user at step 440. For example, the target location may be the neck of the subject. At step 442, the probe acquires ultrasound imaging data and determines a location of the probe on the target location. At step 444 anatomical landmarks are identified in the ultrasound imaging data. In a non-limiting example, the anatomical landmarks include, but are not limited to, thyroid cartilage, CTM, cricoid cartilage, tracheal rings, thyroid gland, and blood vessels. At step 446, a target anatomical landmark is identified, and its location confirmed. In a non-limiting example, the target anatomical landmark is a tracheal ring for insertion of an interventional device according to aspects of the present disclosure. At step 448, the AI guidance software instructs the user to guide the probe to an insertion point. Safety of the insertion of a needle is confirmed at step 450. In a non-limiting example, the safety of the insertion is based on, but not limited to, the path of the needle within the anatomy to reach the target anatomical landmark while avoiding penetrating anatomical structures such as the thyroid gland, unintended blood vessels, bones, or nerves. If it is not safe to inject the needle, then the AI guidance software returns to step 446 to identify and confirm another location of a target anatomical landmark. However, if it is safe to inject the needle, the AI guidance software computes a needle insertion angle and depth and instructs the user to actuate the device to insert the needle at step 452. At step 454, the needle placement may be confirmed by the AI guidance software. If the needle placement is not confirmed, the needle is retracted at step 456 and the AI guidance software returns to step 446 to identify and confirm another location of a target anatomical landmark. If the needle placement is confirmed, then the interventional device is deployed. In a non-limiting example, the interventional device may be, but is not limited to, a wire, dilator, blade, breathing tube, chest tube, vascular catheter, blood clotting agent, or drug.

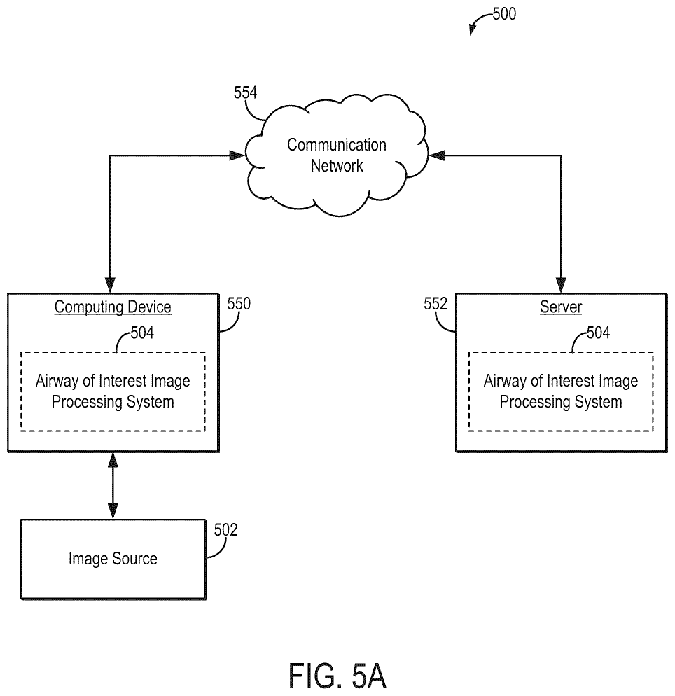

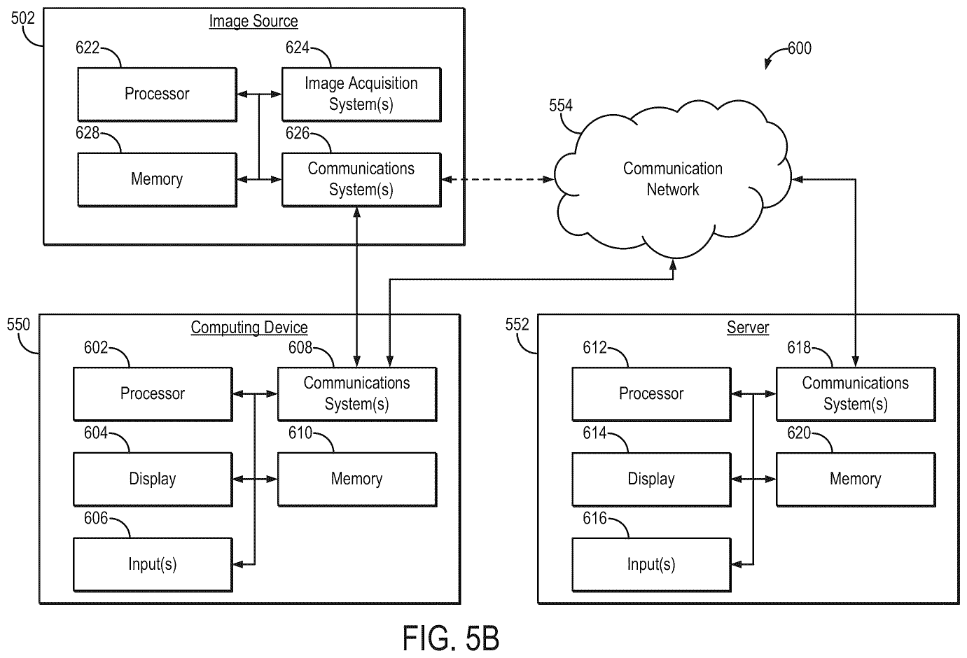

Referring to FIG. 5A, an example of a system 500 for generating and implementing a hybrid machine learning and mechanistic model in accordance with some embodiments of the systems and methods described in the present disclosure is shown. As shown in FIG. 5A, a computing device 550 can receive one or more types of data (e.g., ultrasound, multiparametric MRI data, airway of interest image data, anatomical landmark image data, and the like) from image source 502. In some embodiments, computing device 550 can execute at least a portion of an airway of interest image processing system 504 to generate images of an airway of interest, or otherwise segment an airway of interest from data received from the image source 502.

Additionally or alternatively, in some embodiments, the computing device 550 can communicate information about data received from the image source 502 to a server 552 over a communication network 554, which can execute at least a portion of the airway of interest image processing system 504 to generate images of an airway of interest, or otherwise segment an airway of interest from data received from the image source 502. In such embodiments, the server 552 can return information to the computing device 550 (and/or any other suitable computing device) indicative of an output of the airway of interest image processing system 504 to generate images of an airway of interest, or otherwise segment an airway of interest from data received from the image source 502 that may include use of anatomical landmarks.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on. The computing device 550 and/or server 552 can also reconstruct images from the data.

In some embodiments, image source 502 can be any suitable source of image data (e.g., measurement data, images reconstructed from measurement data), such as an ultrasound system, another computing device (e.g., a server storing image data), and so on. In some embodiments, image source 502 can be local to computing device 550. For example, image source 502 can be incorporated with computing device 550 (e.g., computing device 550 can be configured as part of a device for capturing, scanning, and/or storing images). As another example, image source 502 can be connected to computing device 550 by a cable, a direct wireless link, and so on. Additionally or alternatively, in some embodiments, image source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 5A can each be any suitable communications link or combination of communications links, such as wired links, fiber optic links, Wi-Fi links, Bluetooth links, cellular links, and so on.

Referring now to FIG. 5B, an example of hardware 600 that can be used to implement image source 502, computing device 550, and server 554 in accordance with some embodiments of the systems and methods described in the present disclosure is shown. As shown in FIG. 5B, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, image source 502 can include a processor 622, one or more image acquisition systems 624, one or more communications systems 626, and/or memory 628. In some embodiments, processor 622 can be any suitable hardware processor or combination of processors, such as a CPU, a GPU, and so on. In some embodiments, the one or more image acquisition systems 624 are generally configured to acquire data, images, or both, and can include an ultrasound system. Additionally or alternatively, in some embodiments, one or more image acquisition systems 624 can include any suitable hardware, firmware, and/or software for coupling to and/or controlling operations of an ultrasound system or a subsystem of an ultrasound system. In some embodiments, one or more portions of the one or more image acquisition systems 624 can be removable and/or replaceable.

Note that, although not shown, image source 502 can include any suitable inputs and/or outputs. For example, image source 502 can include input devices and/or sensors that can be used to receive user input, such as a keyboard, a mouse, a touchscreen, a microphone, a trackpad, a trackball, and so on. As another example, image source 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more image acquisition systems 624, and/or receive data from the one or more image acquisition systems 624; to images from data; present content (e.g., images, a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of image source 502. In such embodiments, processor 622 can execute at least a portion of the program to generate images, transmit information and/or content (e.g., data, images) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

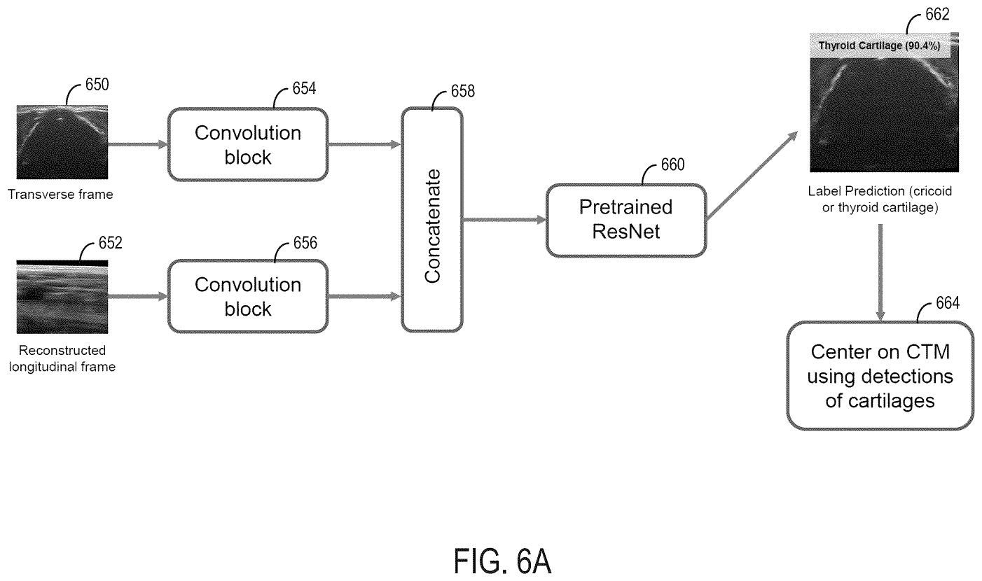

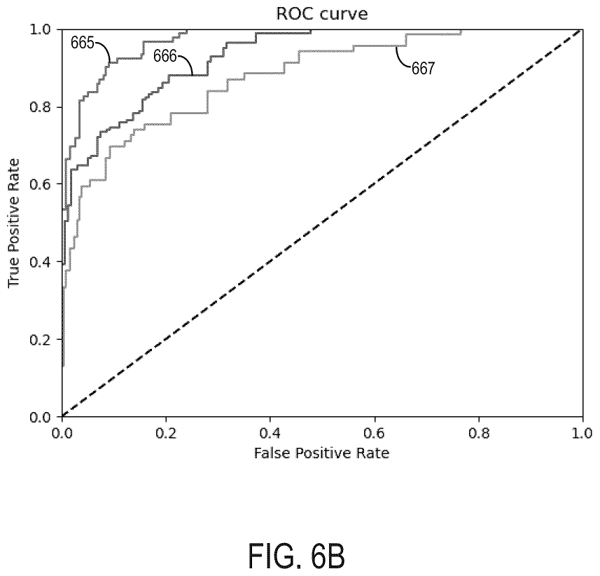

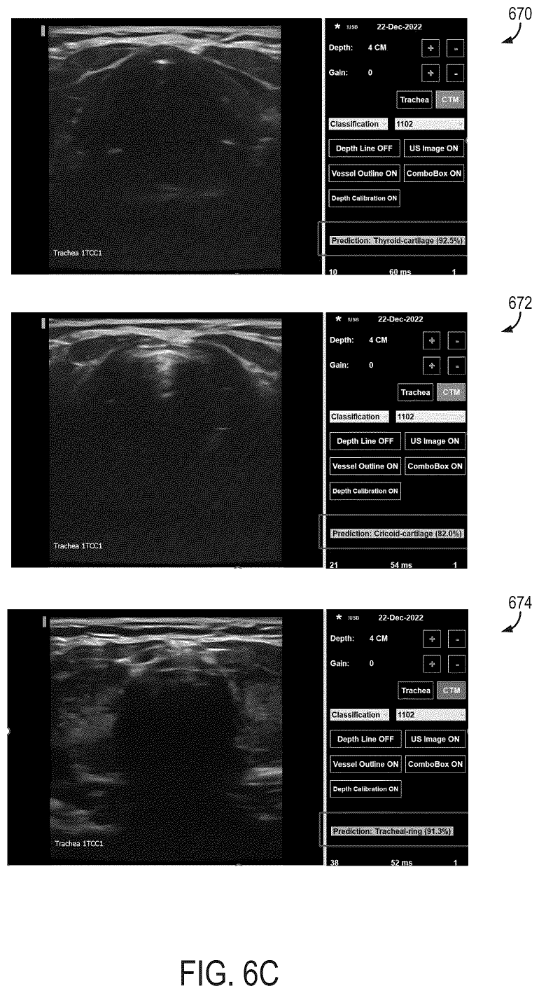

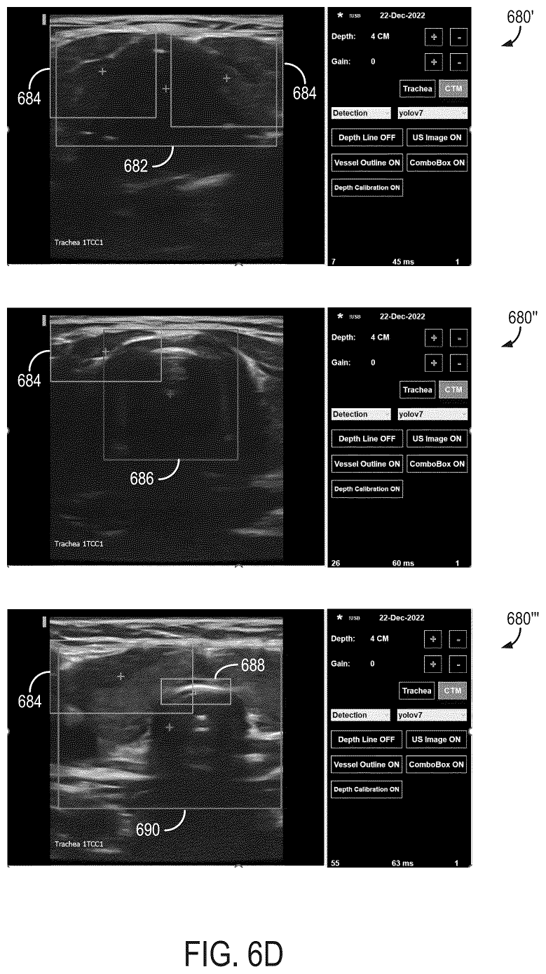

Referring to FIG. 6A, a non-limiting example block diagram of a machine learning or artificial intelligence system is shown. In some configurations, a machine learning or AI system may be in the form of a convolutional neural network (CNN). An ultrasound transverse frame 650 and a reconstructed longitudinal frame 652 may be input to convolutional blocks 654 and 656 respectively, in a CNN architecture. Concatenation layer 658 may concatenate the processed transverse frame 650 and reconstructed longitudinal frame 652. A pretrained RESNET 34 layer 660 may generate a label prediction 662 using the processed transverse frame 650 and reconstructed longitudinal frame 652. Anatomical landmarks may be identified from the labeled predictions in order to identify an airway of interest. In a non-limiting example, the system may center on CTM using cartilage detections at 664 in order to provide guidance for penetrating an airway of interest.

In some configurations, the machine learning or AI system may be configured to determine the anatomical landmarks, identify a location for an airway of interest, and may provide automated guidance for penetrating the airway of interest. The machine learning or AI system may be trained using annotated anatomical images to establish a training for the anatomical landmarks that may be used to determine the location of an airway of interest, and to guide penetration of the airway without impinging upon critical structures to avoid.

In a non-limiting example, a pretrained RESNET AI model 660 may be pretrained using any one of, but not limited to, ImageNet, images from public ultrasound databases, and a custom ultrasound database finetuned for neck ultrasound data. FIG. 6B shows ROC curves for tracheal ring 665 classification (AUC=0.97), thyroid cartilage 666 classification (AUC=0.93), and cricoid cartilage 667 classification (AUC=0.88) according to aspects of the present disclosure. In a non-limiting example, the AI is trained to recognized successive landmarks in the anatomical images including, but not limited to, thyroid cartilage, cricoid cartilage, CTM, tracheal rings, thyroid gland, and strap muscles. FIG. 6C shows example frames during a frame-level classification for gross positioning of the probe on the neck of a subject. The AI predicts the identification of thyroid cartilage 670, cricoid cartilage 672, and tracheal rings 674.

In a non-limiting example, a YOLO (You-only-look-once) bounding box detection AI model may utilize bounding box detection to the anatomical images for precise localization of a needle insertion point. FIG. 6C shows example frames 680′, 680″, and 680′″ as the ultrasound probe is moved on the neck of the subject for bounding box detection of the anatomical images. Frame 680′ includes highlights bounding box 682 identifying the thyroid cartilage and two bounding boxes 684 identifying strap muscles. Frame 680″ shows an additional bounding box 686 identifying cricoid cartilage. Frame 680′″ includes bounding box 688 identifying the CTM or tracheal ring and bounding box 690 identifying the thyroid gland. In accordance with the present disclosure, the CTM or tracheal ring represents the target for an interventional device, while the thyroid gland is an anatomical structure to avoid.

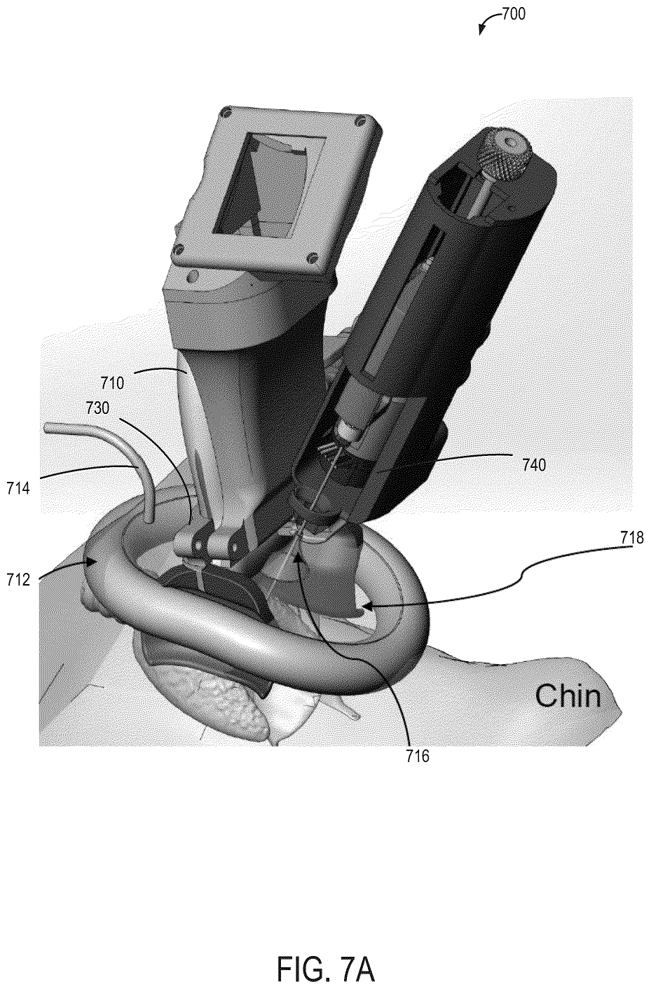

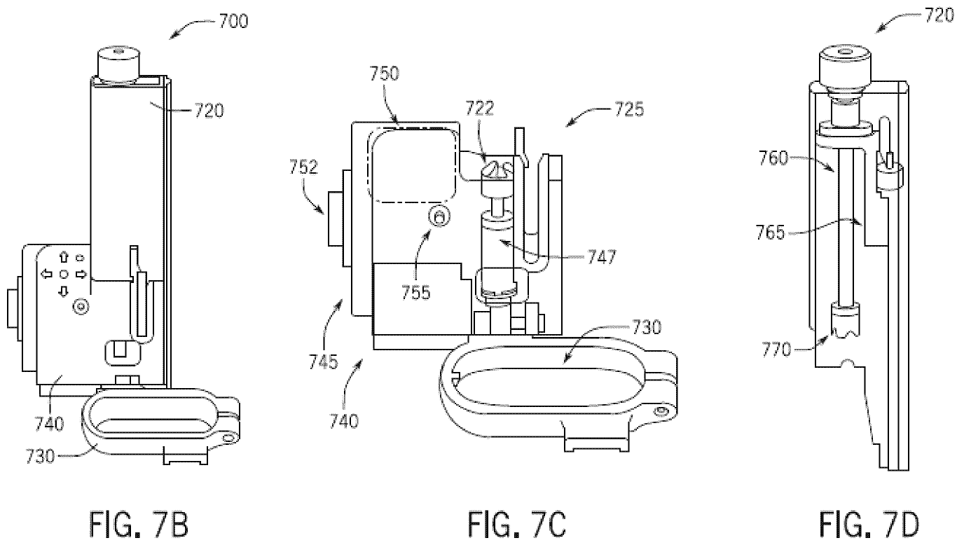

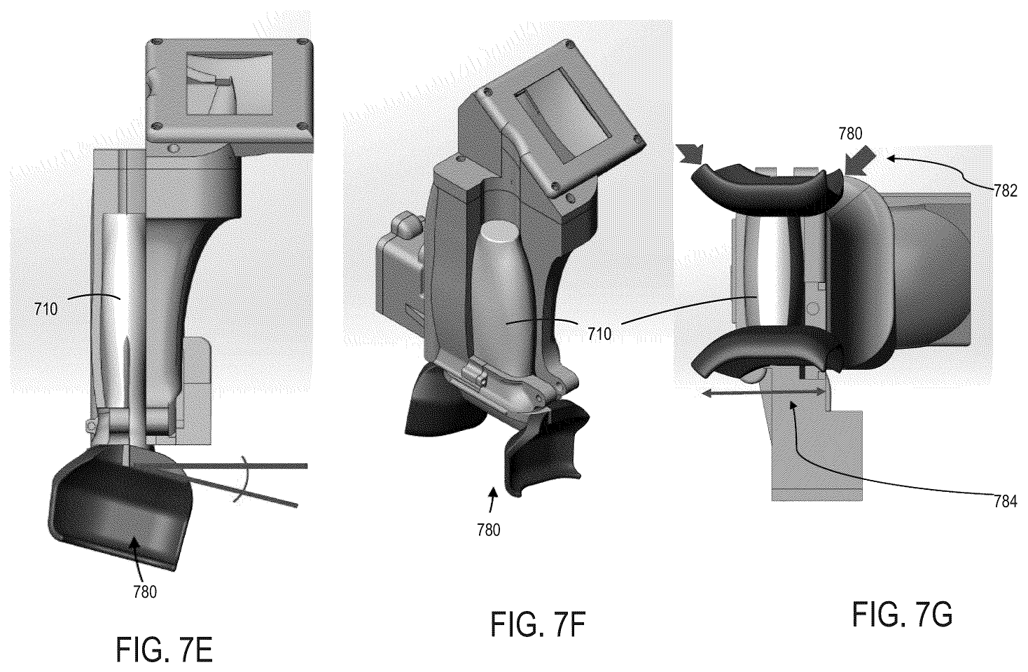

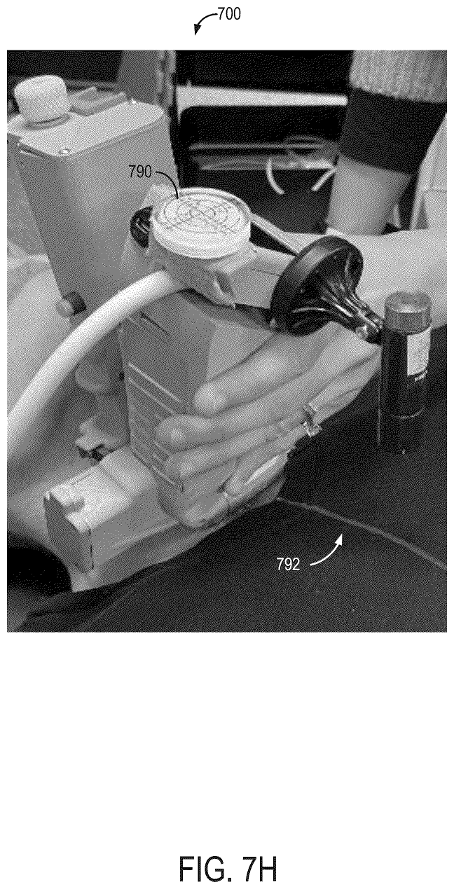

Referring to FIG. 7A, a perspective view of a non-limiting example interventional device guide penetration assembly 700 coupled to an ultrasound probe 710 is shown. Base 740 is shown with ultrasound handle fixture 730 that provides detachable coupling to ultrasound probe 710. The injection assembly 700 may be attached to any ultrasound device, such as by being strapped onto an ultrasound probe 710 using the ultrasound handle fixture 730. Base 740 may include a mechanical support resting on the skin in order to minimize kick-back and improve needle insertion accuracy. A conforming perimeter 712 may be used to protect from aerosolized particles, such as with a use of a port to pump and filter assembly 714. A sealed insertion window 716 with a negative pressure space 718 may be used to ensure a proper seal to prevent aerosolized particles from escaping. A negative pressure barrier may be formed around negative pressure space 718 with an edge along conforming perimeter 712. The port to pump and filter assembly 714 may be used to connect the negative pressure barrier to a pump to form the negative pressure in the negative pressure space 718.

Referring to FIG. 7B, a side view of the interventional device guide injection assembly 700 of FIG. 7A is shown. In a non-limiting example, base 740 contains a motor to set the angle at which the interventional device, which may be a needle, will be inserted. A probe angle relative to vertical may be in a range of 0 to 15 degrees. In a non-limiting example, the probe angle is 15 degrees. The base 740 may also contain a second drive motor to drive the interventional device to the desired depth. The motor may be controlled to vary the insertion speed at different insertion depths, e.g., the needle, cannula or dilator may be inserted relatively slowly through the skin to minimize kick-back and improve accuracy, and then inserted faster subsequently. In some configurations, the drive motor function may be replaced or augmented by a spring or any suitable method of storing mechanical energy, and an additional motor or other suitable method of mechanical actuation to enable injection into a subject. Cartridge 720 is detachably coupled to base 740 and may be configured for the intervention being performed. In non-limiting examples, cartridge 720 may include configurations to treat indications requiring vascular access, tension pneumothorax, or establishing of an airway. Non-limiting example cartridge configurations are listed in Table 1 below.

TABLE 1
Non-limiting example cartridge configurations

| | | Cartridge | |
|---|---|---|---|
| Intervention | | Generation | Capability |
| Vascular | Femoral | 1 | Needle only |
| | Artery/Vein | 2 | Needle with dilator and/or guide wire (or |
| | | | similarly functioning guide) |
| | | 3 | REBOA, clotting agent, other intervention |
| | Internal Jugular | 1 | Needle only |
| | Vein | 2 | Needle with dilator and/or guide wire (or |
| | | | similarly functioning guide) |
| | | 3 | REBOA, clotting agent, other intervention |
| Air | Cricothyrotomy | 1 | Needle only |
| | (or similar | 2 | Breathing tube |
| | methods of | 3 | Breathing tube + forced air |
| | establishing | | |
| | airway access) | | |
| | Tension | 1 | Needle only |
| | Pneumothorax | 2 | Chest tube |
| Abdomen | Ascites | 1 | Needle only |
| | | 2 | Catheter |
| | Bladder | 1 | Needle only |
| | Pregnant | 1 | Needle only |
| | uterus | | |
| | amniocentesis | | |
| Soft | Focal | 1 | Needle only |
| tissue | lesion/tumor | | |
| | biopsy | | |

Referring to FIG. 7C is a side view of the base and ultrasound probe fixture for the interventional device guide of FIG. 7B. Base 740 includes a drive motor 745 to set an insertion angle and/or depth for an interventional device held by cartridge slot 725 coupled by cartridge coupling 722. Advancement motor 747 may be included to advance an interventional device with activation by advancement control 755, which in a non-limiting example is a button. Electrical interface connector 752 may provide communication to an ultrasound imaging system or separate display system. In a non-limiting example, the communication may include the angle for the interventional device, the insertion point location, the location of the target airway, the insertion distance, an indicator of the insertion point location projected proximate to the target airway, or an indicator of the ultrasound probe position at the insertion point location by an illumination display coupled to the system. User guidance signal 750 provide feedback to a user and may take the form or any display intending to direct the user in gross and/or precise placement of the device. In a non-limiting example, user guidance signal 750 includes an arrangement of LEDs. In some configurations, user guidance signal 750 may be coupled to the cartridge 720 and may be specific to the particular indication being treated.

Referring to FIG. 7D is a cross-section of a non-limiting example cartridge 720 compatible with the injection assembly 700 of FIG. 7B. Lead screw 760 may provide for actuation of base coupling 770 to couple the non-limiting example cartridge 720 to base 740 in FIG. 7B. Needle carriage 765 is shown as a non-limiting example of a needle cartridge application.

Referring to FIGS. 7E, 7F and 7G, a side, perspective, and lower views of a non-limiting example interventional device guide penetration assembly is shown, respectively. Ultrasound probe 710 is shown with flexible guide wings 780, which may be set at an angle from the probe 710. The flexible guide wings 780 may be configured to grasp or apply pressure around a patient's anatomy, such as a neck or trachea, to provide for fixation for the assembly, or to aid in localization of the assembly. The flexible guide wings 780 may provide for partial constraint of the anatomy, such as the trachea, and may spread out to accommodate larger patient sizes. Registration may be based off of the muscles in the neck, or the surround anatomy around the target airway of interest. The flexible guide wings 780 may be formed of a flexible or semi-deformable material to automatically adjust to different trachea sizes, yet rigid enough to maintain form without being so stiff that the system may fall away from the surface of the subject. In non-limiting examples, the material used for the flexible guide wings 780 includes rubber, plastic, and the like. In a non-limiting example, the flexible guide wings 780 may be formed of a material with a durometer range of 50-100. In a non-limiting example, the durometer of the material of the guide wings is 92A. The flexible guide wings 780 may include swept edges 782 to enable smooth translation of the assembly along a patient. The flexible guide wings 780 may be formed of a dimension 784 that is long enough to maintain angular stability of the assembly, such as in azimuth, where a cranial-caudal length may aid in providing stability. The flexible guide wings 780 may keep the trachea centered within the view of the ultrasound probe. The flexible guide wings 780 may set the approximate angle of the device relative to the trachea.

Alternatively, an optical stabilizer 790 may be utilized as shown in FIG. 7H instead of the guide wings 790. The optical stabilizer 790 may comprise a level integrated on the housing of interventional device guide penetration assembly 700 and a laser 792 transmitting a visible line across the surface of the subject. In a non-limiting example, the level may be a bubble level or a digital level. In a non-limiting example, the laser may be a transmitted from a light emitting diode (LED).

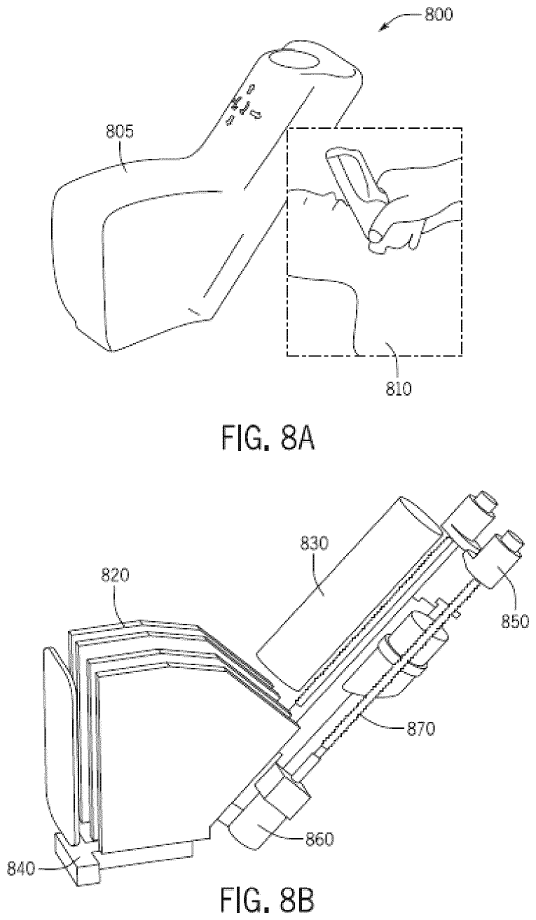

Referring to FIG. 8A, a perspective view of a non-limiting example interventional device guide integrated with an ultrasound probe is shown. Integrated interventional device guide 800 is shown being placed on a subject 810. The integrated interventional device guide 800 may include functionality similar to injection assembly 700 described above with integration with an ultrasound probe. The integrated interventional device guide 800 may be ultrasound guided, and may employ machine learning or artificial intelligence for identifying a target structure for penetration and guiding penetration of the target structure, in accordance with the present disclosure. The integrated ultrasound transducer may provide for excitation, for reading a source, for processing ultrasound signals, and the like. Integrated interventional device guide 800, may include onboard artificial intelligence algorithms, motors, associated drive circuitry, other electronics/mechanics, and the like fit within a housing 805 for the integrated device guide 800. A cartridge, such as described herein, may be detachably coupled to integrated interventional device guide 800. In some configurations, the integrated interventional device guide 800 may be robotically controlled.

Referring to FIG. 8B is an exploded view of the integrated interventional device guide 800 and ultrasound probe of FIG. 8A is shown. Circuit boards 820 may provide for ultrasound guidance from ultrasound transducers 840, and may employ machine learning or artificial intelligence for identifying a target structure for penetration and guiding penetration of the target structure, in accordance with the present disclosure. Battery 830 may provide power for the integrated device. One battery cell is shown in FIG. 8B, but it is to be appreciated that any number of battery cells may be used, such as two cells for extended life, or any other form of power supply. Drivetrain 850 may provide for independent needle or interventional device insertion and cannula insertion. Needle and cannula 870 may be inserted into a subject with motors 860.

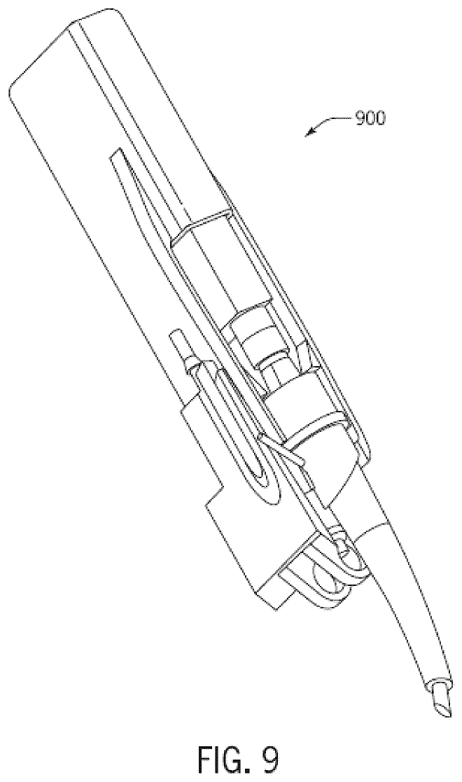

Referring to FIG. 9 is perspective view of a non-limiting example cricothyrotomy cartridge 900 for use in accordance with the present disclosure. As indicated in Table 1 above, different clinical indications may require different types of needles or other hardware/drugs to be introduced into the body. In a non-limiting example, in the case of non-compressible hemorrhage, blood products may need to be rapidly introduced and a needle sheath may provide a path of adequate diameter for rapid introduction of fluid. In another non-limiting example, a catheter may need to be introduced, or a dilating element with larger lumen may be required. Each cartridge may be designed, and clearly labeled with, an intended application. In some configurations, the system may be capable of knowing which type of cartridge device is “plugged” into it. This information may be conveyed through electrical communication between the cartridge and the base, such as radio frequency or direct conducted signals, or through optical communication between the cartridge and the base, or through a mechanical keying specific to the cartridge/base assembly that indicates the cartridge type used, and the like. In a non-limiting example of a mechanical keying, the Femoral Artery/Vein Generation 1 cartridge of Table 1 could be configured such that it depresses a first button in the cartridge slot in the base, whereas the generation 2 cartridge in this family could be configured to depress a second button. In this manner, the base may distinguish between which cartridges have been inserted. In some configurations, the cartridge may be inside of the sterile surgical barrier with the base external to the sterile barrier, such that communication of the cartridge type may be performed through the barrier to ensure safe, effective treatment.

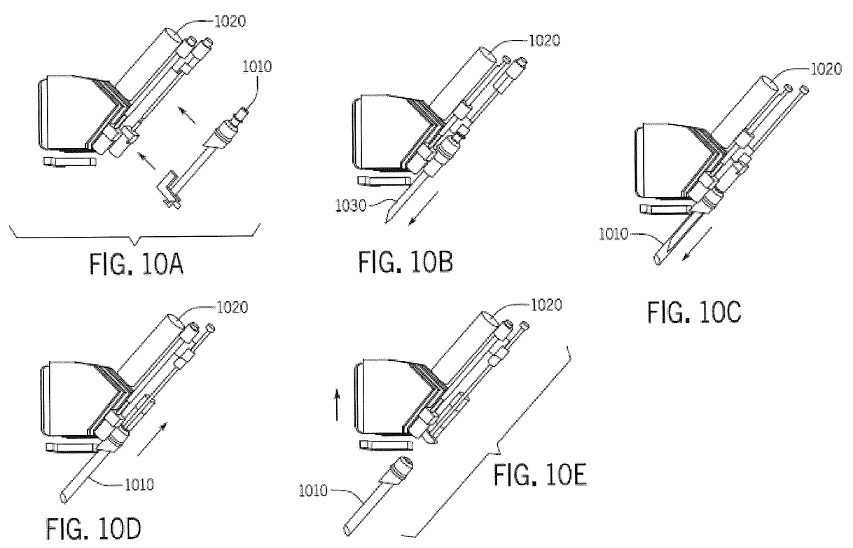

Referring to FIGS. 10A-E, side views of inserting and removing a non-limiting example dilating component into a subject is shown. Some types of cartridges shown in Table 1 may require more than a single step needle insertion process. In a non-limiting example, a cartridge may be configured to install a dilated lumen, which may include a multi-step process. In a non-limiting example, installing a breathing tube through the CTM may include a coaxial assembly consisting of a sharp center element for puncturing and initial path guidance in addition to a coaxial element for dilation and eventual passage of air, which may be introduced according to FIGS. 10A-E.

The sequence shown in FIGS. 10A-10E may be entirely automated by the motors or other mechanical actuation in the system, or may be a combination of automated actuation and human handling. Referring to FIG. 10A, a side views of inserting a non-limiting example dilating component 1010 into a subject is shown. In some configurations, a protector may be removed to insert a disposable version of the dilator 1010 to maintain sterility and safety.

Referring to FIG. 10B a side view of aligning a non-limiting example dilating component 1010 with the interventional device guide 1020 is shown. Needle 1030 may be deployed after device alignment, which may be coaxial with dilating component 1010. In some configurations, the receiving anatomy may be more sensitive to damage or additional mechanical guidance may be required for proper introduction of the larger diameter element. In such configurations, a “guide-wire” device may be used to temporarily protrude from the tip of the inserted assembly, in a function similar to that of the guide-wire used in the Seldinger technique. The “guide-wire” device may be deployed between steps depicted in FIG. 10B and FIG. 10C.

Referring to FIG. 10C, a side view of advancing a non-limiting example dilating component 1010 into the subject is shown. Dilating component 1010 may be advanced over, and may be coaxial with, needle 1030. Dilating component 1010 may provide for expanded access into the subject after insertion. Referring to FIG. 10D, a side view of retracting the needle 1030 from the subject is shown. Referring to FIG. 10E, a side view of removing the interventional device guide 1020 is show where dilating component 1010 is retained in the subject and may be used for access from an interventional device.

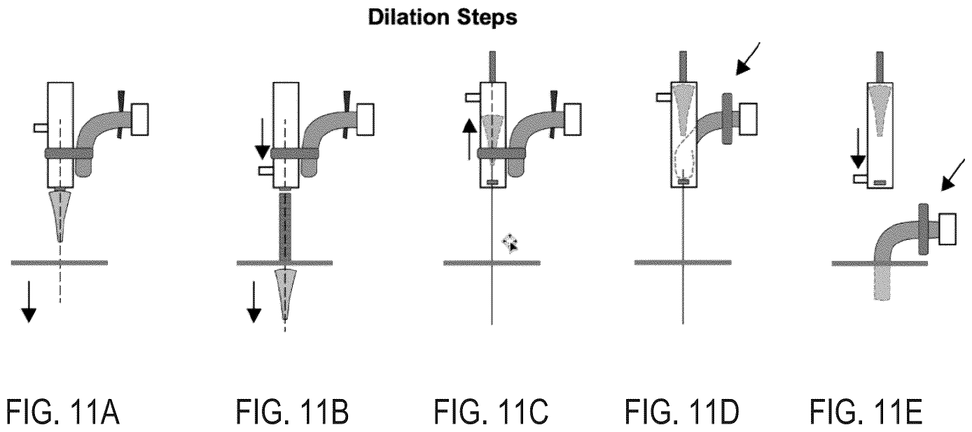

Referring to FIGS. 11A-11E, non-limiting example steps for a method of dilation in accordance with the present disclosure are shown. FIGS. 11A-11E may provide a method for concomitant dilation and tracheostomy tube placement. FIG. 11A depicts needle/cannula advancement. Semi-autonomous or autonomous needle placement into the trachea may be performed using a machine learning or AI system to identify anatomical landmarks and a target airway. Upon confirmation of device placement, a user may engage the tracheostomy placement. In some configurations, an incision may be made to relieve pressure on the skin surface of the subject before any dilation takes place or prior to any advancement of any penetrating devices into the subject. FIG. 11B depicts dilation advancement. An autonomous dilation sheath may be advanced along the shaft, piercing the skin permitting adequate dilation and continuing through the anterior tracheal wall. A safety stop may be used to prevent over advancement of dilation system. FIG. 11C depicts dilator retraction. A dilation apparatus may autonomously retract into the system, leaving in place a distal dilator track for the tracheostomy tube, and to maintain a track into the airway. FIG. 11D depicts where trach is engaged. An attachment of the tracheostomy tube may engage the track pathway, and may autonomously carry the tracheostomy into position. FIG. 11E depicts where a trach is advanced. The system may release with tracheostomy tube freely available for connection to an anesthesia circuit.

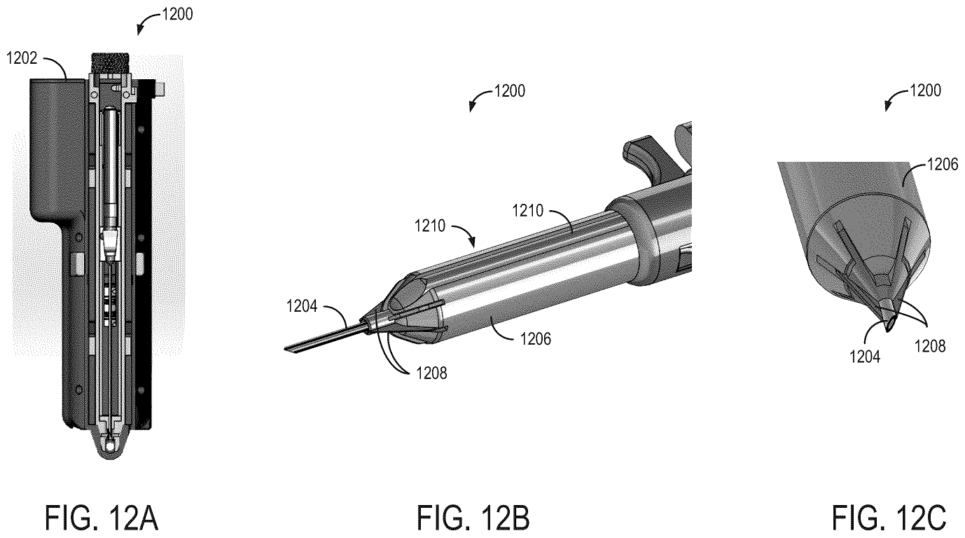

FIGS. 12A-12C show a non-limiting example of an interventional device according to aspects of the present disclosure 1200. FIG. 12A shows the interventional device within a cartridge 1202 as described in any of the previous embodiments. FIG. 12B shows a close-up view of the interventional device including a needle 1204, dilator 1206, microsurgical blades 1208, and a catheter 1210 within a trough 1212 along the dilator 1206. In a non-limiting example, the dilator may have an outer diameter of 11 mm. FIG. 12C shows a front view of a distal end of the interventional device 1200. In a non-limiting example, the needle 1204 and blades 1208 are combined to create a single cut into which the dilator pushes.

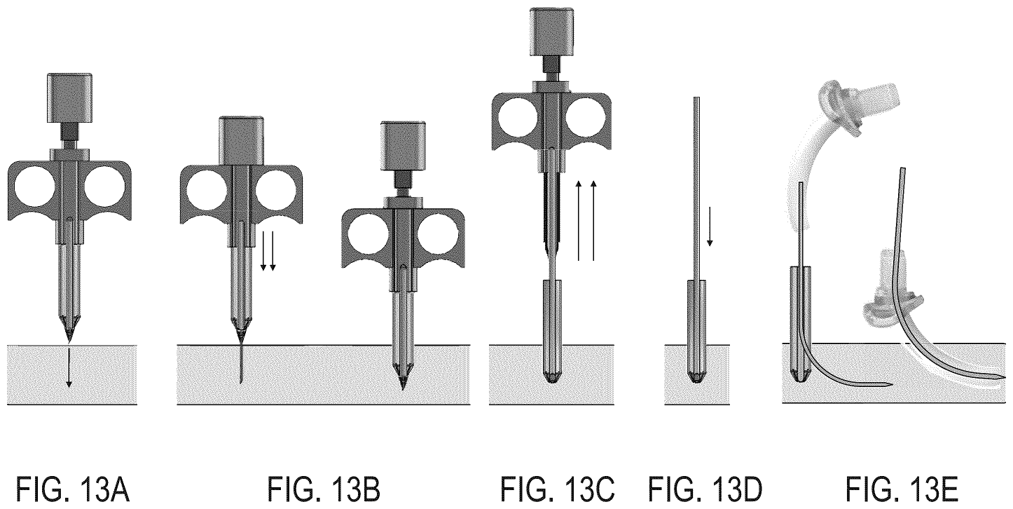

FIGS. 13A-13E depict non-limiting example steps for a method of dilation in accordance with the interventional device 1200 from FIGS. 12A-12C. FIGS. 13A-13E may provide a method for concomitant dilation and tracheostomy tube placement. FIG. 13A depicts needle/cannula advancement. Semi-autonomous or autonomous needle placement into the trachea may be performed using a machine learning or AI system to identify anatomical landmarks and a target airway. Upon confirmation of device placement, a user may engage the tracheostomy placement. FIG. 13B depicts the advancement of blades and a dilator along the needle shaft toward the tip of the needle. The blade makes an incision to relieve pressure on the skin surface. An autonomous dilation sheath may be advanced along the shaft, piercing the skin permitting adequate dilation and continuing through the anterior tracheal wall. A safety stop may be used to prevent over advancement of dilation system. FIG. 13C depicts the retraction of the needle and blades, leavening the dilator in the trachea in FIG. 13D. The dilator may serve as a temporary tracheostomy tube or converted to a permanent tracheostomy tube. FIG. 13D depicts the advancement of a catheter through a trough into the trachea. In a non-limiting example, the trough is oriented such that the catheter curves into the trachea at at 90°. At FIG. 13E the dilator may be removed and an endotracheal tube is inserted over the catheter and may be autonomously carried into position. The system may release with tracheostomy tube freely available for connection to an anesthesia circuit.

The present disclosure has described one or more preferred embodiments, and it should be appreciated that many equivalents, alternatives, variations, and modifications, aside from those expressly stated, are possible and within the scope of the invention.

1. A system for guiding an interventional device in an interventional procedure of a subject, comprising:
an ultrasound probe;
a guide system coupled to the ultrasound probe and configured to guide the interventional device into a field of view (FOV) of the ultrasound probe;
a non-transitory memory having instructions stored thereon;
a processor configured to access the non-transitory memory and execute the instructions, wherein the processor is caused to:
access image data acquired from the subject using the ultrasound probe, wherein the image data include at least one image of an anatomical landmark structure of the subject;
determine, from the image data and the anatomical landmark structure, a location of a target airway within the subject;
determine an insertion point location for the interventional device based upon the location of the target airway and guide placement of the ultrasound probe to position the guide system at the insertion point location; and
track the interventional device from the insertion point location to the target airway.

2. The system of claim 1, wherein the anatomical landmark structure includes at least one of: thyroid cartilage, cricothyroid membrane (CTM), cricoid cartilage, thyroid gland, or tracheal rings.

3. The system of claim 1, wherein the processor is further caused to determine at least one of an angle for the interventional device from the insertion point location to the target airway, a rotational angle for the ultrasound probe with respect to the subject, or an insertion distance from the insertion point location to the target airway based upon the anatomical landmark structure.

4. The system of claim 3, further comprising a display system and wherein the processor is further configured to cause the display system to show at least one of the angle for the interventional device, the insertion point location, the location of the target airway, the insertion distance, an indicator of the insertion point location projected proximate to the target airway, or an indicator of the ultrasound probe position at the insertion point location by an illumination display coupled to the system.

5. The system of claim 1, wherein the processor is further caused to track the interventional device from the insertion point location to the target airway and provide real-time feedback to a user based on tracking the interventional device.

6. The system of claim 1, wherein the processor is configured to receive a plurality of images of the anatomical landmark structure of the subject acquired in real-time to access the image data.

7. The system of claim 6, wherein the plurality of images includes a plurality of views of the target airway, and wherein the processor is configured to assess the plurality of images of the anatomical landmark structure and the plurality of views of the target airway to identify a location on the subject where the interventional device reaches the target airway from the insertion point location without penetrating a landmark to avoid in the subject.

8. The system of claim 7, wherein the landmark to avoid includes at least one of a bone, an unintended blood vessel, a non-target organ, or a nerve.

9. The system of claim 7, wherein the plurality of images includes images at a plurality of different timeframes.

10. The system of claim 1, wherein the guide system includes a removable cartridge coupled to a base of the guide system, wherein the cartridge contains the interventional device.

11. The system of claim 10, wherein the interventional device is at least one of a needle, wire, dilator, blade, breathing tube, chest tube, vascular catheter, blood clotting agent, or drug.

12. The system of claim 11, wherein the interventional device is configured to perform at least one of cricothyrotomy or tracheotomy.

13. The system of claim 1, wherein the guide system is detachably coupled to the ultrasound probe with an ultrasound handle fixture.

14. The system of claim 1, wherein the guide system is coupled to the ultrasound probe by integration with the ultrasound probe in a housing.

15. The system of claim 14, wherein the guide system includes a power supply.

16. The system of claim 1, wherein the guide system is configured to guide the interventional device automatically.

17. The system of claim 1, wherein the processor is further caused to determine if the target airway has been penetrated by determining the presence of CO₂ using a CO₂ sensor.

18. The system of claim 1, further comprising flexible wings to provide localization for the interventional device by grasping a surface region around the anatomical landmark structure of the subject.

19. The system of claim 18, wherein the wings include a material of durometer 92A.

20. The system of claim 1, further comprising a negative pressure barrier to isolate the interventional device from a user or the subject.

21. The system of claim 1, wherein the processor is further caused to input the image data acquired from the subject into an artificial intelligence (AI) model to identify anatomical landmark structures in the image data.

22. The system of claim 21, wherein the AI model further outputs one or more bounding boxes identifying the anatomical landmark structures in the image data.

23. The system of claim 22, wherein the AI model is trained using a You-only-look-once (YOLO) deep learning network for outputting the one or more bounding boxes.

24. The system of claim 21, wherein the AI model is trained using a pretrained ResNet deep learning network.

25. A method of performing an interventional procedure on a subject, the method comprising:
accessing image data acquired from the subject using the ultrasound probe, wherein the image data include at least one image of an anatomical landmark structure of the subject;
determining, from the image data and the anatomical landmark structure, a location of a target airway within the subject;
determining an insertion point location for the interventional device based upon the location of the target airway and guide placement of the ultrasound probe to position the guide system at the insertion point location; and
tracking the interventional device from the insertion point location to the target airway.
